(19) United States
(12) Patent Application Publication
(10) Pub. No.: US 2021/0238535 A1
(43) Pub. Date: Aug. 5, 2021

(54) AUTOMATED PRODUCTION OF CAR-EXPRESSING CELLS

(71) Applicant: Inscripta, Inc., Boulder, CO (US)

(72) Inventor: Christopher Abraham, Boulder, CO (US)

(21) Appl. No.: 17/123,077

(22) Filed: Dec. 15, 2020

Related U.S. Application Data
(60) Provisional application No. 62/969,144, filed on Feb. 2, 2020.

(51) Int. Cl. C12M 1/36; C12M 3/00; C12M 1/26; C12N 15/90; C12N 9/22; C12N 15/11; C12N 5/0783

(52) U.S. Cl. CPC C12M 41/48; C12M 23/44; C12M 33/00; C12N 15/907; C12N 2510/00; C12N 15/11; C12N 5/0636; C12N 5/0646; C12N 2310/20; C12N 9/22

(57) ABSTRACT

In an illustrative embodiment, the present disclosure provides methods, systems and/or instruments for the automated editing of immune cells for chimeric antigen receptor therapies.

### RELATED CASES

The present application claims priority to U.S. Ser. No. 62/969,144, filed 2 Feb. 2020.

### FIELD OF THE INVENTION

The present disclosure relates to methods and systems for introducing genome edits to living cells for expression of chimeric antigen receptors in T and NKcells.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Genome editing with engineered nucleases is a method in which changes to nucleic acids are made in the genome of a living organism. Certain nucleases create site-specific double-strand breaks at target regions in the genome, which can be repaired by nonhomologous end-joining or homologous recombination, resulting in targeted edits.

Recently, T cells and NK cells have been genetically engineered to produce artificial cell receptors on their surface called chimeric antigen receptors, or CARs. CARs are proteins that allow immune cells to recognize a specific, pre-selected protein or antigen found on targeted tumor cells. For example, autologous CAR-expressing T cells for treatment of blood cancers can be created by obtaining live T cells from a patient, introducing a construct for expression of a CAR to the T cells, optionally culturing and expanding the cells in the laboratory, then re-infusing the modified T cells to a patient. Through the guidance of the engineered cell receptor, CAR-expressing T cells can recognize and destroy cancer cells that display the specific antigen on their surfaces.

In 2017, two chimeric antigen receptor T (CAR-T) cell-based immunotherapy products were approved. KYMRIAH® has been approved for the treatment of acute lymphoblastic leukemia, and YESCARTA® has been approved for treatment of lymphoma, To date, the use of CAR-T outside blood cancers has been limited.

There is thus a need for instruments, systems, modules and methods for improving the production and use of CAR-expressing cell therapies. The present disclosure addresses this need.

### SUMMARY OF ILLUSTRATIVE EMBODIMENTS

In an illustrative embodiment, the present disclosure provides methods, systems and/or instruments for the automated editing of immune cells for chimeric antigen receptor therapies.

The disclosure provides methods, systems and/or instruments for creating CAR-expressing cells in an automated system. In preferred aspects, the automated system for producing the CAR-expressing immune cells is a closed system, minimizing the risk of contamination or human error in the production of such cell populations. The present disclosure thus provides methods of creating the CAR-expressing cells in a closed system without human intervention from introduction of the cells to be edited into an instrument through collection of the edited, CAR-expressing cells.

The CAR-expressing cells of the disclosure may be edited to express one or more CARs on their surface, the CARs comprising an extracellular domain which specifically binds a predetermined antigen. In specific aspects, the disclosure provides methods of creating and using CAR-expressing cell therapies that express two or more different CARs, either on the same cells or on different cells within the modified cell population. The editing of the cell populations is carried out in an automated fashion.

In some aspects, the disclosure provides a chimeric antigen receptor (CAR)-expressing mammalian immune cell population created using an automated multi-module editing instrument for nuclease-directed genome editing, wherein the instrument comprises a housing, a receptacle configured to receive cells and one or more rationally designed nucleic acids comprising sequences to facilitate nuclease-directed genome editing events in the cells, a growth module, a module for introduction of the nucleic acid(s) into the cells, an editing module for allowing the nuclease-directed genome editing events to occur in the cells, and a processor-based system configured to operate the instrument based on user input. The nuclease-directed genome editing events created by the automated instrument result in a mammalian cell population comprising individual immune cells expressing one or more CARs. For examples of closed system, multi-module cell editing instruments, see U.S. Pat. Nos. 10,253,316; 10,329,559; 10,323,242; 10,421,959; 10,465,185; 10,519,437; 10,584,333; 10,584,334; 10,647,982; 10,689,645; 10,738,301; 10,738,663 and U.S. Ser. Nos. 16/412,175 and 16/988,694, all of which are herein incorporated by reference in their entirety. In addition to cell and nucleic acid receptacles and growth, transformation (i.e., transfection or transduction) and editing modules, there may be present additional modules such as a cell concentration module, one or more selection modules and/or a cell storage module. Further, some modules may be combined physically, that is, the growth module may also serve as the editing module and/or the editing module may also be the selection module. The transformation module may be a separate module, such as an electroporation module or the transformation module may be a transduction module, which is combined with the growth, selection and editing modules.

In some aspects, the disclosure provides a chimeric antigen receptor (CAR)-expressing mammalian immune cell population created using an automated multi-module cell editing instrument for nuclease-directed genome editing, wherein the instrument comprises a housing, a cell receptacle configured to receive cells, a nucleic acid receptacle configured to receive one or more rationally designed nucleic acids comprising sequences to facilitate nuclease-directed genome editing events in the cells, a growth module, a module for introduction of the nucleic acid(s) into the cells, and an editing module for allowing the nuclease-directed genome editing events to occur in the cells. The nuclease-directed genome editing events created by the automated instrument result in a mammalian cell population comprising individual immune cells expressing one or more CARs.

In some aspects, the nuclease-directed genome editing events in the mammalian immune cells creates a clonal cell population expressing a single CAR. In some aspects, the nuclease-directed genome editing events in the mammalian immune cells creates a clonal cell population of individual cells expressing two or more CARs. In some aspects, the nuclease-directed genome editing events in the mammalian immune cells creates a cell population with individual cells expressing one or more different CARs.

In some aspects, the mammalian immune cells are T cells. In some aspects, the mammalian immune cells are natural killer cells.

In some aspects, the cells are treated using the methods of the disclosure to remove the components of the editing system prior to the further editing of the cells in the population. The disclosure provides methods, systems and/or instruments for creating CAR-expressing cells in an automated multi-module system. In preferred aspects, the multi-module automated system for producing the CAR-expressing immune cells is a closed system, minimizing the risk of contamination or human error in the production of such cell populations. The present disclosure thus provides methods of creating the CAR-expressing cells via automated multi-module cell editing in a closed system without human intervention from introduction of the cells to be edited into an instrument through collection of the edited, CAR-expressing cells.

Thus some embodiments comprise a method for creating a chimeric antigen receptor (CAR)-expressing mammalian cell population using an automated editing instrument for nuclease-directed genome editing, comprising the steps of: providing an automated multi-module cell processing instrument comprising: a housing; a first receptacle configured to receive cells and a second receptacle configured to receive one or more rationally designed nucleic acids comprising a coding sequence for a nucleic acid-guided nuclease and a gRNA and donor DNA sequence to be transcribed; a growth module; a transformation module; an editing module; and a processor and liquid handling system configured to move the mammalian cell population from the first receptacle to the growth module; to more the mammalian cell population from the growth module to the transformation module; to move the one or more rationally designed nucleic acids from the second receptacle to the transformation module; and to move the mammalian cell population from the transformation module to the editing module; providing the mammalian cell population to the receptacle to receive cells; providing the one or more rationally designed nucleic acids to the receptacle to receive the one or more rationally designed nucleic acids; transferring the mammalian cell population from the receptacle to receive cells to the growth module; growing the mammalian cell population; transferring the mammalian cell population from the growth module to the transformation module; transferring the one or more rationally designed nucleic acids from the receptacle to receive the one or more rationally designed nucleic acids to the transformation module; transforming the mammalian cell population with the one or more rationally designed nucleic acids comprising a coding sequence for the nucleic acid-guided nuclease and the gRNA and donor DNA sequence to be transcribed to produce transformed mammalian cells; transferring the transformed mammalian cells to the editing module; and allowing the transformed mammalian cells to edit, resulting in a mammalian cell population comprising cells expressing one or more CARs.

In some embodiments, the nucleic acid-guided nuclease is replace with a nickase-RT fusion protein and the gRNA and donor DNA sequence to be transcribed are replaced with a CF gRNA.

In some aspects, the mammalian cell population comprising cells expressing one or more CARs express a single CAR, in some aspects, the mammalian cell population comprising cells expressing one or more CARs express two or more CARs, and in some aspects, the mammalian cell population comprising cells expressing one or more CARs is a cell population with individual cells expressing one or more different CARs.

In some aspects, the instrument further comprises a nucleic acid assembly module, and in some aspects, the nucleic acid assembly module is a module performing isothermal nucleic acid assembly.

In some aspects, the mammalian cell population comprising cells expressing one or more CARs are autologous and in some aspects, the mammalian cell population comprising cells expressing one or more CARs are allogeneic.

In some aspects, the growth module and the editing module are combined into one module and in some aspects, the growth module and the editing module are different modules.

In some aspects, the automated multi-module cell processing instrument further comprises a selection module, where in some aspects, the selection module is separate from the editing module. In other aspects, the selection module and the editing module are combined.

In some aspects, the automated multi-module cell processing instrument further comprises a cell concentration module.

The CAR-expressing cells of the disclosure may be edited to express one or more CARs on their surface, the CARs comprising an extracellular domain which specifically binds a predetermined antigen. In specific aspects, the disclosure provides methods of creating and using CAR-expressing cell therapies that express two or more different CARs, either on the same cells or on different cells within the modified cell population.

In certain aspects, the cells to be edited for expression of one or more CARs are autologous immune cells taken from the patient and edited to express one or more CARs using the methods, systems and/or instruments of the disclosure. In some aspects, the cells be edited for expression of the one or more CARs are allogenic immune cells, optionally matched to a patient or patients to reduce rejection or other adverse events. In some aspects, the cells to be edited for expression of the one or more CARs are immune cells modified to be donor cells that can work with one or more patient populations. In some aspects, the cells to be edited for expression of the one or more CARs are immune cells modified to be “universal donor” cells that can work across patient populations.

Instruments and systems of the disclosure can also be used to provide automated methods to edit stem cells that can be further differentiated into immune cells, e.g., pluripotent mammalian stem cells, hematopoietic stem cells, induced pluripotent stem cells and the like. Examples of such methods that can be modified for use in the automated multi-module instrument of the disclosure include US Pub. 2018/0250339.

In various embodiments, the CAR-expressing cells produced using the methods, systems and/or instruments of the invention are introduced into a patient in a therapeutically effective amount for treatment of a disorder, e.g. a cancer that expresses one or more predetermined antigen. Preferably, the delivery is parenteral delivery. In specific aspects, the CAR-expressing cells are provided in a pharmaceutically acceptable excipient which supports maintenance of the cells. Suitable buffers and salts are well known in the art for maintenance and administration of various cell populations, as will be apparent to one of skill in the art upon reading the disclosure. The patient can be any mammal in need of such treatment, and preferably the patient is human.

In a specific aspect, the present disclosure relates to methods for preparing a CAR-expressing cell population of immune cells expressing one or more chimeric antigen receptor (CAR) following editing of the cells. Specific methods comprise: contacting in vitro one or more immune cells that have been modified to express the CAR with a stimulus that induces expansion of the immune cells to provide an expanded immune cell population; editing the nucleic acids of the immune cells using the automated methods and systems to provide immune cells expressing designed CARs. The cells can be activated prior to or following the editing operation, e.g., activating in vitro an edited T cell population to produce a CAR-expressing effector T cell population. In certain methods, the T cells in the resulting effector T cell population are preferably attenuated for proliferation.

It is to be understood that the invention is not limited to the specific details and components set forth in the following description. The invention is capable of embodiments in addition to those described and of being practiced and carried out in various ways. As such, those skilled in the art will appreciate that the conception upon which this disclosure is based may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

In specific aspects, the disclosure provides methods of creating multiple edits to the immune cells or other cell population to introduce two or more edits to the individual cells. Preferably, this recursive editing involves the removal of one or more components of the editing system such as by curing one or more of the components of the editing system, followed by subjecting the cells to a further editing operation. Such “recursive” editing using the methods of the disclosure may include subjecting the cells to two, three, four, five, six, seven, eight, nine, ten or more editing operations with intervening treatment to remove one or more editing components from the previous operation. This allows the cells to undergo sequential introduction of components of a directed cell editing system to introduce multiple CARs or other edits to the CAR-expressing cells without interference of the components of the directed cell editing system of a previous editing operation.

In some aspects, the cells are treated using the methods of the disclosure to remove one or more components of the editing system following a single edit and before introduction to a patient. Such “curing” allows the cells to be free of editing machinery prior to introduction of the cells to the patient, and/or allows the edited cells to be subjected to recursive editing operations without the interference of the components of a prior editing operation. For additional information regarding integrated instrumentation related to recursive and curing methods see U.S. Pat. Nos. 10,421,959 and 10,738,663.

In other aspects, the methods of the disclosure to remove one or more components of the editing system following final editing of recursive editing operations. This enriches for cells that contain the directed edit(s) but which do not have an operational editing system and/or components thereof remaining in the cells. A cell population created with this final clearing step is enriched for cells containing the directed edit(s) but without the potential interference of editing components upon further analysis and/or experimentation.

The removal of the editing components of an introduced editing system may be inducible, e.g., induced by temperature, expression of a nuclease, etc. For additional information regarding integrated instrumentation and methods for inducible editing, see, e.g., U.S. Pat. Nos. 10,329,559; 10,669,559; 10,550,363; 10,633,626; and 10,760,043.

In some aspects, the editing component removal is provided in a single plasmid system used for introducing CARs into immune cells. In other aspects, the editing component removal system is provided using a system comprising two or more plasmids. In yet other aspects, the nucleic acid-guided nuclease or nickase-RT fusion enzyme may be introduced to the cells to be edited as a protein, with the guide nucleic acid and donor DNA expressed from a vector.

In some aspects, the curing of a vector in a living cell is carried out using temperature. In other aspects, the curing of a vector in a living cell is carried out using a nuclease. In specific aspects, the curing of a vector in a living cell is carried out using a CRISPR nuclease which targets the editing plasmid or vector. In other specific aspects, the curing of a vector in a living cell is carried out using an endonuclease which cleaves the vector, and preferably an endonuclease which has exonuclease activity.

In some aspects, the editing plasmid is designed to introduce a single directed edit for introduction of CARs into immune cells of a cell population in a single editing operation. This population of immune cells may be a clonal population were each cell expresses the same CAR per cell, or the edited cell population may include a cell population where the cells express different CARs.

In other aspects, the editing plasmid is designed to introduce two or more directed edits into each cell in a cell population in a single editing operation. In certain aspects, vectors delivering two (or more) directed edits per cell may be used in a recursive editing system to provide multiple combinatorial editing, e.g., creation of multiple edits in each single cell of an edited cell population to provide a population of cells with multiple CAR edits introduced.

Other features, advantages, and aspects will be described below in more detail.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W.H. Freeman Pub., New York, N.Y.; all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The terms used herein are intended to have the plain and ordinary meaning as understood by those of ordinary skill in the art. The following definitions are intended to aid the reader in understanding the present disclosure, but are not intended to vary or otherwise limit the meaning of such terms unless specifically indicated.

The term “chimeric antigen receptor” and CAR as used interchangeably herein refers to a receptor that is not generally present on a native cell type to be edited and that binds specifically to a predetermined antigen.

The term “combinatorial editing” as used herein refers to one or more editing operations to introduce two or more edits into a cell. Combinatorial editing is intended to include methods and systems for introducing two or more edits to a cell in a single editing operation as well as introducing two or more edits to a cell using a recursive method or system.

The term “curing” as used herein is the elimination of one or more nucleic acid-guided editing components required for editing the genome of a cell. In some aspects, curing refers to the removal of one or more vectors that contain nucleic acid-guided components required for editing the genome of a cell.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. The donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

As used herein, the phrase “engine vector” comprises a coding sequence for a nucleic acid-guided nuclease (e.g., CRISPR nuclease) or nickase-RT fusion enzyme to be used in the nucleic acid-guided nuclease systems and methods of the present disclosure. Engine vectors also typically comprise a selectable marker. As used herein the phrase “editing vector” comprises, when utilizing a nucleic acid-guided nuclease (e.g., a CRISPR nuclease), a donor nucleic acid, including an alteration to the cellular target sequence that prevents nuclease binding at a PAM or spacer in the cellular target sequence after editing has taken place, and a coding sequence for a gRNA. The editing vector comprises, when utilizing a nuclease-RT fusion protein, a CREATE fusion gRNA” or “CF gRNA.” The editing vector may also comprise a selectable marker and/or a barcode. In some embodiments, the engine vector and editing vector may be combined; that is, all nucleic acid-guided editing components may be found on a single vector. Further, the engine and editing vectors comprise control sequences operably linked to, e.g., the nucleic acid-guided nuclease or nickase-RT fusion coding sequence, donor nucleic acid, guide nucleic acid(s), and selectable marker(s).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease. The terms “CREATE fusion gRNA” or “CF gRNA” refer to a gRNA engineered to function with a nucleic acid-guided nickase/reverse transcriptase fusion protein (a “nickase-RT fusion”).

The terms “natural killer cells” or “NK cells” refer to differentiated lymphocytes with a CD16+CD56+ and/or CD57+ TCR-phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release cytokines that stimulate or inhibit the immune response.

As used herein, a “nucleic acid-guided nuclease” refers to a CRISPR nuclease, such as MAD7, Cas9, Cpf1 or Cas12 nucleases. As used herein, “nucleic acid-guided nickase/reverse transcriptase fusion” or “nickase-RT fusion” refers to a nucleic acid-guided nickase or nucleic acid-guided nuclease or CRISPR nuclease that has been engineered to act as a nickase rather than a nuclease that initiates double-stranded DNA breaks fused to a reverse transcriptase, which is an enzyme used to generate cDNA from an RNA template. For information regarding nickase-RT fusions see, e.g., U.S. Pat. No. 10,689,669 and U.S. Ser. No. 16/740,421.

“Nucleic acid-guided editing components” refers to an nucleic acid-guided nuclease or a nickase-RT fusion; and a guide nucleic acid and donor DNA in the case of a nucleic acid-guided nuclease and a CF gRNA in the case of a nickase-RT fusion.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible.

The term “recursive” as used herein refers to a process that is performed in an iterative fashion. The recursive systems and methods of the disclosure are performed on a multi-module instrument.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The term “T cells” refers to differentiated lymphocytes with a CD34+, T cell receptor (TCR)+ having either CD4+ or CD8+ phenotype. The T cells may be effector T cells or a regulatory T cells.

As used therein the term “transformation” is used generally for all manner of introducing a nucleic acid or protein into a desired cell type, including transfection and transduction.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like.

### The Invention in General

The present disclosure provides compositions and methods for enhancing the editing efficiency of editing methods and systems using nucleic acid-directed nucleases or nickase-RT fusions. The methods, systems and/or instruments of the disclosure can be used in the production of cell therapies comprising one or more chimeric antigen receptors (“CARs”). These oligonucleotides are preferably rationally designed to introduce specific, designed edits to the genomes of cells in a cell population, as described in more detail herein.

The present disclosure has many advantages over the conventional CAR technology that is currently in therapeutic use and/or development. First, multiple CARs can be introduced to a cell population, either with single cells in a population expressing different CARs or as individual cells expressing one or more CARs. The cells to be edited can be autologous immune cells, allogeneic immune cells, or a “donor” immune cell population. This allows targeting of various diseases, including but not limited to cancers, infectious diseases such as with HIV, and undesired immune responses such as autoimmunity and transplant rejection.

Although in some aspects the disclosure describes the editing of a cell population to produce cells expressing a single, rationally-designed CAR, the ability to produce cell populations expressing two to thousands more CARs expands the potential uses of the technology to other disease states, including infectious diseases, cancers having heterogenous cell populations, and autoimmune diseases.

### Chimeric Antigen Receptors

Chimeric antigen receptors (“CARs”) are cell-surface receptor proteins that have been engineered to give immune cells (e.g., T cells or NK cells) the new ability to target a specific antigen, e.g. a tumor specific antigen (TSA).

Normal proteins in the body are not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

Specific examples of TSAs that can be targeted using the CAR-T cell-based therapy of the invention include, but are not limited to, products of mutated oncogenes and tumor suppressor genes; products of other mutated genes that promote tumor growth, e.g., angiogenesis factors; overexpressed or aberrantly expressed cellular proteins; tumor antigens produced by oncogenic viruses; oncofetal antigens; altered cell surface glycolipids and glycoproteins; and cell type-specific differentiation antigens.

CAR-expressing T and NK cells express antigen receptors against tumor-associated surface antigens, thus redirecting the effector cells and enhancing tumor-specific immunosurveillance.

In some aspects, the CAR-expressing immune cells can target a predetermined antigen that is present on the cell surface of cells associated with an autoimmune disorder and/or transplantation rejection.

In some aspects, the CAR-expressing immune cells can target a predetermined antigen that is present or predicted to be present on an infectious agent.

Certain predetermined antigens may be expressed on healthy tissues, potentially leading to off tumor/on target toxicity by CAR-engineered cells. Accordingly, to control such potentially severe side effects, various additional edits or nuclease-directed editing designs can be used to limit the serious adverse events that could be associated with the use of particular CAR-expressing immune cells. For example, the insertion of suicide genes into CAR-modified effectors can provide a means for efficient depletion of these cells. See, e.g., U.S. Pat. Nos. 5,586,153; 6,066,624; 6,537,805; and 6,989,268. In another example, such as taught in US Pub. No. 2018/0251789, the editing machinery introduced to a cell comprises a synthetic regulatory system with a multifunctional nucleic acid-directed nuclease and at least two distinct gRNAs, wherein the synthetic regulatory system modulates cleavage and transcription in a mammalian cell such as a human cell. The system in some aspects acts as a safety switch to control the expression of the CARs.

### T Cells Modified to Express CARs

CAR-T cell therapy uses T cells engineered with CARs for, e.g., treatment of cancer, autoimmune disease, infectious disease and the like. For CAR-T immunotherapy, T cells are modified to recognize cancer cells in order to more effectively target and destroy them. Scientists harvest T cells from people, genetically alter them, then infuse the resulting CAR-T cells into patients to attack their tumors. CAR-T cells can be either derived from T cells in a patient's own blood (autologous) or derived from the T cells of another healthy donor (allogenic). Once isolated from a person, these T cells are genetically engineered to express a CAR that specifically binds an antigen on the cell-surface of a cancer of interest, Preferably, CAR-T cells are engineered to be specific to a tumor specific antigen, and thus not targeting healthy cells.

Basic principles for CAR-T design can be found, e.g., in Sadelain, et al., Cancer Discovery, 3(4): 388-98 (2013); and US Pub. Nos. 2018/0265585 and 2018/0186878. CARs for use with CAR-T therapies preferably combine both antigen-binding and T-cell activating functions into a single receptor.

After CAR-T cells are infused into a patient, the CAR-T cells come in contact with their targeted antigen on a cell, bind to it, become activated, then proceed to proliferate and become cytotoxic. CAR-T cells destroy the antigen-presenting cells through several mechanisms, including extensive stimulated cell proliferation, increasing the degree to which they are cytotoxic, and by causing the increased secretion of factors that can affect other cells such as cytokines, interleukins, and growth factors. See, e.g., Hartmann, et al., EMBO Molecular Medicine, 9 (9):1183-1197 (2017); and Tang, et al., Oncotarget, 6 (42): 44179-90 (2015).

For T cells, engagement of the CD4⁺ and CD8⁺ T cell receptor (TCR) alone is not sufficient to induce persistent activation of resting naive or memory T cells. Fully functional, productive T cell activation requires a second co-stimulatory signal from a competent antigen-presenting cell (APC). Co-stimulation is achieved naturally by the interaction of the co-stimulatory cell surface receptor on T cells, known as CD28, with the appropriate counter-receptors on the surface of the APC, known as CD80and CD86. An APC is normally a cell of host origin which displays a moiety which will cause the stimulation of an immune response. APCs include monocyte/macrophages, dendritic cells (DCs), B cells, and any number of virally-infected or tumor cells which express a protein on their surface recognized by T cells, and can also be used for the antigen-dependent activation of T cells.

In some aspects, the disclosure is directed to the production and use of CAR expressing T cells which have been modified to limit their proliferation within the recipient. This can be accomplished, e.g., through the introduction of adducts into the genomic nucleic acids of CAR-T cell-derived effector cells following expansion in vitro, which prevent further division of the expanded and activated CAR-T cell-derived effector cells. Because some degree of cytokine release is likely a necessary consequence of T cell activation and therefore efficacy of CAR-T cell-based therapy, the adducts are introduced with a frequency necessary to prevent cell division (and so further T cell proliferation), but that permits the CAR-T cell-derived effector cells to retain immunologic function (e.g., complete immunologic function, partial immunologic function), including the expression of one or more effector cytokines. In some aspects, the present disclosure relates to a CAR-T cell-derived effector cell population that is attenuated for proliferation. The cell population comprises a population of activated T cells expressing a chimeric antigen receptor (CAR), the CAR comprising an extracellular domain which specifically binds a predetermined targeted antigen. The nucleic acids of the activated T cells have been modified by reaction with a nucleic acid targeting compound that reacts directly with the nucleic acid (e.g., modified by reaction with a nucleic acid targeting compound). In certain embodiments, the activated T cells are attenuated for proliferation due to the introduction in crosslinks within the cell's nucleic acid. Preferably, these crosslinks include inter-strand crosslinks in the cell's genomic DNA.

In some aspects, the present disclosure provides methods of inducing a T-cell response to at least one predetermined antigen in a subject, comprising administering to the subject a CAR-T cell-derived effector cell population produced using the automated methods, systems and/or instruments of the disclosure, as described herein. The CAR-T cell-derived effector cell population is provided to a mammal, and preferably a human, in an amount sufficient to induce an anti-tumor response to a cancer in the subject, wherein the cancer expresses the predetermined antigen. In this specific aspect, the predetermined antigen is preferably a tumor-specific antigen.

In a specific aspect, the present disclosure relates to methods for preparing a CAR-T cell-derived effector cell population comprising a population of activated T cells expressing one or more CARs, the CAR comprising an extracellular domain which specifically binds a predetermined antigen, and where the CAR-T cell-derived effector cell population is attenuated for proliferation. The CAR-T cells of the present disclosure can optionally be expanded and activated in vitro prior to administration to a patient to provide a sufficient CAR-T cell-derived effector cell population. The activation step, however, may precede or follow modification of the nucleic acid expressing the CARs.

In some embodiments, the stimulus that induces expansion of the T cells can be a non-specific expansion stimulus, and the expanded T cell population may be subsequently activated by contacting the T cells in the expanded T cell population with the predetermined antigen under conditions in which the T cells are activated. In these latter embodiments, the T cells in the expanded T cell population may be attenuated for proliferation either before or following the activation step.

In another example, the CAR-expressing cells can be modified and/or introduced with another therapeutic to inhibit certain serious adverse events such as cytokine release syndrome (“CRS”). See e.g., US Pub. No. 2018/0228866. CRS is a common and potentially lethal complication of CAR-T cell therapy. CRS is a non-antigen specific toxicity that can occur as a result of the high-levels of CAR-T cell expansion and immune activation typically required to mediate clinical benefit using modern immunotherapies such as CAR-T cell transfer. Timing of symptom onset and CRS severity depends on the inducing agent and the magnitude of immune cell activation. Symptom onset typically occurs days to occasionally weeks after T cell infusion, coinciding with maximal in vivo T-cell expansion. In recent reports of CRS following adoptive T-cell therapy for cancer, the incidence and severity of the syndrome is greater when patients have large tumor burdens, due to the expression of production of proinflammatory cytokines such as TNF-α by the adoptively-transferred, expanding and activated CAR-T cell populations.

### Natural Killer Cells Edited to Express CARs

Natural killer (NK) cells mediate anti-cancer effects and have shown a lower risk of inducing graft-versus-host disease (GvHD) than CAR-modified T cells as CAR-expressing NK cells are short-lived effector cells. Furthermore, regulatory requirements for modified NK cells have been established as a new field in cellular immunotherapy. See, e.g., Liu, et al., Protein Cell, 8(12):861-877 (2017) and Glienke, et al., Front Pharmacol., 6:21 (2015). The unique biology of NK cells allows them to serve as a safe, effective, alternative immunotherapeutic strategy to CAR-modified T cells in the clinic. The formation of the immunological synapse between CAR-modified T or NK cells and their susceptible target cells is known to be essential. The role of the immunological synapse in CAR T and NK cell immunotherapies allows scientists to harness the power of CAR-modified T and NK cells to treat cancer and infectious diseases. CAR-modified NK cells have applicability in the treatment of, e.g., cancers and infectious disease. Adoptive cell-based therapy using CAR-modified NK cells has the potential to extend the survival of cancer patients by enhancing the antitumor effectiveness of CAR-modified cells

Basic principles for CAR-NK design and expansion can be found in, e.g., Rezvani, et al., Mol Ther., 2;25(8):1769-1781(2017); Lin, et al., Biochim Biophys Acta Rev Cancer, 1869(2):200-215 (2018); and US Pub. No. 2018/0245044.

### Nuclease-Directed Genome Editing

In some embodiments, the compositions and methods described herein are employed to perform nucleic acid-guided nuclease editing (e.g., CRISPR or RNA-guided nuclease editing) to introduce desired edits to a population of cells. The most commonly employed method for using nucleic acid-guided nucleases to introduce precision edits it to co-deliver an appropriate gRNA and donor DNA with the donor DNA serving as a template for homology-directed repair (HDR) at the intended site. Nucleic acid-guided nuclease editing begins with a nucleic acid-guided nuclease complexing with an appropriate synthetic guide nucleic acid in a cell which can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may reside within an editing cassette and under the control of a constitutive or an inducible promoter as described below. For additional information regarding “CREATE” editing cassettes comprising a gRNA and a covalently-linked donor DNA, see U.S. Pat. Nos. 9,982,278; 10,266,849; 10,240,167; 10,351,877; 10,364,442; 10,435,715; and 10,465,207 and U.S. Ser. Nos. 16/551,517; 16,773,618; and 16,773,712, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20nucleotides long, or 15, 16, 17, 18, 19, or 20 nucleotides in length.

In the present methods and compositions, the guide nucleic acids are provided as a sequence to be expressed from a plasmid or vector and comprises both the guide sequence and the scaffold sequence as a single transcript under the control of an inducible promoter. The guide nucleic acids are engineered to target a desired target sequence (either cellular target sequence or curing target sequence) by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, an intron, a PAM, or “junk” DNA) or a curing target sequence in an editing vector.

As stated above, the editing guide nucleic acid may be and preferably is part of an editing cassette that encodes the donor nucleic acid that targets a cellular target sequence. Alternatively, the editing guide nucleic acid may not be part of the editing cassette and instead may be encoded on the editing vector backbone. For example, a sequence coding for an editing guide nucleic acid can be assembled or inserted into a vector backbone first, followed by insertion of the donor nucleic acid in, e.g., an editing cassette. In other cases, the donor nucleic acid in, e.g., an editing cassette can be inserted or assembled into a vector backbone first, followed by insertion of the sequence coding for the editing guide nucleic acid. Preferably, the sequence encoding the editing guide nucleic acid and the donor nucleic acid are located together in a rationally designed editing cassette and are simultaneously inserted or assembled into a vector backbone to create an editing vector. In yet other embodiments, the sequence encoding the guide nucleic acid and the sequence encoding the donor nucleic acid are both included in the editing cassette.

The target sequence is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In certain embodiments, the genome editing of a cellular target sequence both introduces a desired DNA change to a cellular target sequence, e.g., the genomic DNA of a cell, and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate. The range of target sequences (both cellular target sequences and curing target sequences) that nucleic acid-guided nucleases can recognize is constrained by the need for a specific PAM to be located near the desired target sequence. As a result, it often can be difficult to target edits with the precision that is necessary for genome editing.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/Cpf1, MAD2, or MAD7 or other MADzymes and nickase-RT fusions thereof. Nickase-RT enzymes typically comprise a CRISPR nucleic acid-guided nuclease engineered to cut one DNA strand in the target DNA rather than making a double-stranded cut, and the nickase portion is fused to a reverse transcriptase. For more information on nickase-RT editing see U.S. Ser. Nos. 16/740,418; 16/740,420 and 16/740,421, all filed 11 Jan. 2020. As with the guide nucleic acid, the nuclease (either nucleic acid-guided nuclease or nickase-RT fusion) may be encoded by a DNA sequence on a vector (e.g., the engine vector) and be under the control of a promoter, which in some embodiments is an inducible promoter. In some embodiments, the inducible promoter may be separate from but the same as the inducible promoter controlling transcription of the guide nucleic acid; that is, a separate inducible promoter drives the transcription of the nucleic acid-guided nuclease or nickase-RT fusion and guide nucleic acid sequences but the two inducible promoters may be the same type of inducible promoter. Alternatively, the inducible promoter controlling expression of the nuclease may be different from the inducible promoter controlling transcription of the guide nucleic acid. In yet other embodiments, the nucleic acid-guided nuclease or nickase-RT fusion enzyme may be delivered to the cells as a protein.

Another component of the nucleic acid-guided nuclease system is the donor nucleic acid comprising homology to the cellular target sequence. In some embodiments, the donor nucleic acid is on the same polynucleotide (e.g., editing vector or editing cassette) as the guide nucleic acid and preferably is (but not necessarily is) under the control of the same promoter as the editing gRNA (e.g., a single promoter driving the transcription of both the editing gRNA and the donor nucleic acid). The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. The donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence.

Again, the donor nucleic acid is preferably provided as part of a rationally-designed editing cassette, which is inserted into an editing vector backbone where the editing vector backbone may comprise a promoter driving transcription of the editing gRNA and the donor DNA, and also comprise a selectable marker different from the selectable marker contained on the engine vector, as well as a curing target sequence that is cut or cleaved during curing. Moreover, there may be more than one, e.g., two, three, four, or more editing gRNA/donor nucleic acid rationally-designed editing cassettes inserted into an editing vector (alternatively, a single rationally-designed editing cassette may comprise two to several editing gRNA/donor DNA pairs), where each editing gRNA is under the control of separate different promoters, separate like promoters, or where all gRNAs/donor nucleic acid pairs are under the control of a single promoter. In preferred embodiments the promoter driving transcription of the editing gRNA and the donor nucleic acid (or driving more than one editing gRNA/donor nucleic acid pair) is an inducible promoter and the promoter driving transcription of the nuclease or nickase-RT fusion is an inducible promoter as well. In some embodiments and preferably, the nuclease and editing gRNA/donor DNA are under the control of the same inducible promoter.

Inducible editing is advantageous in that singulated cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. See U.S. Pat. Nos. 10,550,363; 10,633,626; 10,760,043; and 10,723,995 for methods and compositions related to induction and normalization.

In addition to the donor nucleic acid, an editing cassette may comprise and preferably does comprise one or more primer sites. The primer sites can be used to amplify the editing cassette by using oligonucleotide primers; for example, if the primer sites flank one or more of the other components of the editing cassette.

Also, as described above, the donor nucleic acid may comprise—in addition to the at least one mutation relative to a cellular target sequence—one or more PAM sequence alterations that mutate, delete or render inactive the PAM site in the cellular target sequence. The PAM sequence alteration in the cellular target sequence renders the PAM site “immune” to the nucleic acid-guided nuclease and protects the cellular target sequence from further editing in subsequent rounds of editing if the same nuclease is used.

In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library editing gRNAs and of donor nucleic acids representing, e.g., gene-wide or genome-wide libraries of editing gRNAs and donor nucleic acids. The library of editing cassettes is cloned into vector backbones where, e.g., each different donor nucleic acid is associated with a different barcode.

Additionally, in some embodiments, an expression vector or cassette encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs. In some embodiments, the engineered nuclease comprises NLSs at or near the amino-terminus, NLSs at or near the carboxy-terminus, or a combination.

The engine and editing vectors comprise control sequences operably linked to the component sequences to be transcribed. As stated above, the promoters driving transcription of one or more components of the nucleic acid-guided nuclease editing system preferably are inducible. A number of gene regulation control systems have been developed for the controlled expression of genes in plant, microbe, and animal cells, including mammalian cells, including the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

In alternative embodiments, the compositions and methods described herein are employed to perform nucleic acid-guided nuclease editing (e.g., RNA-guided nuclease editing) to introduce desired edits to a population of cells; however, the compositions and methods described herein employ a nucleic acid-guided nickase/reverse transcriptase fusion protein (“nickase-RT” fusion) as opposed to a nucleic acid-guided nuclease (i.e., a “CRISPR nuclease”). The nickase-RT fusion employed herein differs from “traditional” CRISPR editing in that instead of initiating double-strand breaks in the target genome, the nickase initiates a nick in a single strand of the target genome. Further, the fusion of the nickase to a reverse transcriptase eliminates the need for a donor DNA. Instead, a CF gRNA (defined supra) is used where the reverse transcriptase portion of the nickase-RT fusion protein incorporates the desired edit into the cellular genome. See, e.g., U.S. Pat. No. 10,689,669; and U.S. Ser. Nos. 16/740,420 and 16/740,421.

### Delivery of Nucleic Acid-Guided Editing Components to T Cells and NK Cells

Nucleic acid-guided editing components can be delivered as DNA or RNA or the nucleic acid-guided nuclease or nickase-RT fusion may be delivered to the cells as a protein. Conventional viral and non-viral based gene transfer methods can be used to introduce nucleic acids in cells, such as mammalian cells, or target tissues. Such methods can be used to administer nucleic acids encoding components of a nucleic acid-guided nuclease system to cells in culture, or in a host organism. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and 4,897,355; and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024.

The preparation of lipid:nucleic acid complexes, including targeted liposomes such as immunolipid complexes, is well known to one of skill in the art (see, e.g., Crystal, Science, 270:404-410 (1995); Blaese, et al., Cancer Gene Ther., 2:291-297 (1995); Behr, et al., Bioconjugate Chem., 5:382-389 (1994); Remy, et al., Bioconjugate Chem., 5:647-654 (1994); Gao, et al., Gene Therapy 2:710-722 (1995); Ahmad, et al., 52:4817-4820 (1992); and U.S. Pat. Nos. 4,186,183, 4,217,344, 4,235,871, 4,261,975, 4,485,054, 4,501,728, 4,774,085, 4,837,028, and 4,946,787).

Alternatively, a viral delivery system based on any appropriate virus may be used to deliver the nucleic acid-guided editing components to mammalian cells. Alternatively, hybrid viral systems may be of use. The choice of viral delivery system will depend on various parameters, such as transduction efficiency of the system, pathogenicity, immunological and toxicity concerns, and the like. In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

For example, in one embodiment, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells.

Another viral delivery system useful with the nucleic acid-guided editing components is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and-be passed from parent cell to progeny cells as a stably-integrated component of the host genome.

Additionally, Anelloviridae are a recently-discovered family of viruses, classified as vertebrate viruses and have a non-enveloped capsid, which is round with isometric, icosahedral symmetry. The Anelloviridae genome is not segmented and contains a single molecule of circular, negative-sense, single-stranded DNA. The complete genome is 3000-4000 nucleotides long. Anellovirus species are highly prevalent and genetically diverse, causing chronic human viral infections that have not yet been associated with disease. At least 200 different species are present in humans and animals.

In some embodiments, lentiviruses are the preferred members of the retrovirus family for use in the present embodiment. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visna-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization. The main advantage to the use of lentiviral vectors is that gene transfer is persistent in most cell types.

Adenoviruses are a relatively well-characterized homogenous group of viruses, including over 50 serotypes. Adenoviruses are medium-sized (90-100 nm), nonenveloped (without an outer lipid bilayer) icosahedral viruses composed of a nucleocapsid and a double-stranded linear DNA genome. There are 57 described serotypes in humans, which are responsible for 5-10% of upper respiratory infections in children, and many infections in adults as well. Adenoviruses are classified as group I under the Baltimore classification scheme, meaning their genomes consist of double-stranded DNA, and are the largest nonenveloped viruses. Because of their large size, they are able to be transported through the endosome (i.e., envelope fusion is not necessary). The virion also has a unique “spike” or fiber associated with each penton base of the capsid that aids in attachment to the host cell via the coxsackie-adenovirus receptor on the surface of the host cell.

The adenovirus genome is a linear, non-segmented double-stranded (ds) DNA that is between 26 and 45 kb, allowing the virus to theoretically carry 22 to 40 genes.

Although this is significantly larger than other viruses in its Baltimore group, adenovirus is still a very simple virus and is heavily reliant on the host cell for survival and replication. Once the virus has successfully gained entry into the host cell, the endosome acidifies, which alters virus topology by causing capsid components to disassociate. With the help of cellular microtubules, the virus is transported to the nuclear pore complex, where the adenovirus particle disassembles. Viral DNA is subsequently released, which can enter the nucleus via the nuclear pore. After this, the DNA associates with histone molecules; thus, viral gene expression can occur and new virus particles can be generated. Unlike lentiviruses, adenoviral DNA does not integrate into the genome.

Other viral or non-viral systems known to those skilled in the art also may be used to deliver the nucleic acid-guided editing components to the cells of interest, including but not limited to gene-deleted adenovirus-transposon vectors that stably maintain virus-encoded transgenes in vivo through integration into host cells; systems derived from Sindbis virus or Semliki forest virus; or systems derived from Newcastle disease virus or Sendai virus.

Before transduction into the mammalian cells, the viral vector is packaged into viral particles. Any method known in the art may be used to produce infectious viral particles comprising a copy of the viral editing cassette delivery vector. Generally, there are two alternative methods for packaging the editing cassette vector viral particles for delivery. One method utilizes packaging cells that stably express in trans the viral proteins that are required for the incorporation of the viral editing cassette vector into viral particles, as well as other sequences necessary or preferred for a particular viral delivery system (for example, sequences needed for replication, structural proteins and viral assembly) and either viral-derived or artificial ligands for tissue entry. The packaging cells then replicate viral sequences, express viral proteins and package the viral editing cassette vectors into infectious viral particles.

Alternatively, a cell line that does not stably express necessary viral proteins may be co-transfected with two or more constructs to achieve efficient production of functional particles. One of the constructs comprises the viral editing cassette vector, and the other construct(s) comprises nucleic acids encoding the proteins necessary to allow the cells to produce functional virus (replication and packaging construct) as well as other helper functions. After production in a packaging cell line, the viral particles containing the viral editing cassette vectors are purified and quantified (titered). Purification strategies include density gradient centrifugation, or, preferably, column chromatographic methods.

Once produced, the virus particles are delivered to the cells in 3D culture at an MOI of approximately <0.1 (e.g., in a Poisson-limited manner). Post-transduction, a selective agent such as an antibiotic is added to the medium to enrich for cells that have been transduced; alternatively, the viral editing cassette vector may comprise a marker for magnetic selection. Because antibiotic selection for mammalian cells can take up to a week of growth in culture, magnetic or FACS selection is preferred at this step. After enrichment, the cells are allowed to grow and recover and then are dissociated and transformed with an engine plasmid comprising a coding sequence for the nucleic acid-guided nuclease or nickase fusion or with the nuclease or nickases fusion protein itself. After a suitable time for editing and cell recovery, the cells are again enriched, by, e.g., FACS sorting, a magnetic marker (e.g., a second, different marker if magnetic selection was used after cellular transduction), or antibiotic.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Fully-Automated Singleplex RGN-directed Editing Run

Singleplex automated genomic editing using MAD7 nuclease was successfully performed with an automated multi-module instrument of the disclosure. See U.S. Pat. No. 9,982,279. The performance was using an automated instrument as set forth in U.S. Ser. No. 16/024,816. These documents are incorporated by reference herein for all purposes.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The transformation module comprised an ADP-EPC cuvette. See, e.g., U.S. Pat. No. 62/551069. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The paramters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁻⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example II: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “ lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The transformation module comprised an ADP-EPC cuvette. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in the isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

I claim:

1. A method for creating a chimeric antigen receptor (CAR)-expressing mammalian cell population using an automated editing instrument for nuclease-directed genome editing, comprising the steps of:
providing an automated multi-module cell processing instrument comprising:
a housing;
a first receptacle configured to receive cells and a second receptacle configured to receive one or more rationally designed nucleic acids comprising a coding sequence for a nucleic acid-guided nuclease and a gRNA and donor DNA sequence to be transcribed;
a growth module;
a transformation module;
an editing module; and
a processor and liquid handling system configured to move the mammalian cell population from the first receptacle to the growth module; to more the mammalian cell population from the growth module to the transformation module; to move the one or more rationally designed nucleic acids from the second receptacle to the transformation module; and to move the mammalian cell population from the transformation module to the editing module;
providing the mammalian cell population to the receptacle to receive cells;
providing the one or more rationally designed nucleic acids to the receptacle to receive the one or more rationally designed nucleic acids;
transferring the mammalian cell population from the receptacle to receive cells to the growth module;
growing the mammalian cell population;
transferring the mammalian cell population from the growth module to the transformation module;
transferring the one or more rationally designed nucleic acids from the receptacle to receive the one or more rationally designed nucleic acids to the transformation module;
transforming the mammalian cell population with the one or more rationally designed nucleic acids comprising a coding sequence for the nucleic acid-guided nuclease and the gRNA and donor DNA sequence to be transcribed to produce transformed mammalian cells;
transferring the transformed mammalian cells to the editing module; and
allowing the transformed mammalian cells to edit, resulting in a mammalian cell population comprising cells expressing one or more CARs.

2. The automated method of claim 1, wherein the mammalian cell population comprising cells expressing one or more CARs express a single CAR.

3. The automated method of claim 1, wherein the mammalian cell population comprising cells expressing one or more CARs express two or more CARs.

4. The automated method of claim 1, wherein the mammalian cell population comprising cells expressing one or more CARs is a cell population with individual cells expressing one or more different CARs.

5. The automated method of claim 1, wherein the mammalian cell population comprising cells expressing one or more CARs are T cells.

6. The automated method of claim 1, wherein the mammalian cell population comprising cells expressing one or more CARs are natural killer cells.

7. The automated method of claim 1, wherein the instrument further comprises a nucleic acid assembly module.

8. The automated method of claim 7, wherein the nucleic acid assembly module is a module performing isothermal nucleic acid assembly.

9. The automated method of claim 1, wherein the mammalian cell population comprising cells expressing one or more CARs are autologous.

10. The automated method of claim 1, wherein the mammalian cell population comprising cells expressing one or more CARs are allogeneic.

11. The automated method of claim 1, wherein the growth module and the editing module are combined into one module.

12. The automated method of claim 1, wherein the growth module and the editing module are different modules.

13. The automated method of claim 1, further comprising a selection module.

14. The automated method of claim 1, wherein the selection module is separate from the editing module.

15. The automated method of claim 1, wherein the selection module and the editing module are combined.

16. The automated method of claim 1, further comprising a cell concentration module.

17. A method for creating a chimeric antigen receptor (CAR)-expressing mammalian cell population using an automated editing instrument for nuclease-directed genome editing, comprising the steps of:
providing an automated multi-module cell processing instrument comprising:
a housing;
a first receptacle configured to receive cells and a second receptacle configured to receive one or more rationally designed nucleic acids comprising a coding sequence for a nickase-RT fusion protein and a CF gRNA sequence to be transcribed;
a growth module;
a transformation module;
an editing module; and
a processor and liquid handling system configured to move the mammalian cell population from the first receptacle to the growth module; to more the mammalian cell population from the growth module to the transformation module; to move the one or more rationally designed nucleic acids from the second receptacle to the transformation module; and to move the mammalian cell population from the transformation module to the editing module;
providing the mammalian cell population to the receptacle to receive cells;
providing the one or more rationally designed nucleic acids to the receptacle to receive the one or more rationally designed nucleic acids;
transferring the mammalian cell population from the receptacle to receive cells to the growth module;
growing the mammalian cell population;
transferring the mammalian cell population from the growth module to the transformation module;
transferring the one or more rationally designed nucleic acids from the receptacle to receive the one or more rationally designed nucleic acids to the transformation module;
transforming the mammalian cell population with the one or more rationally designed nucleic acids comprising a coding sequence for the nickase-RT fusion protein and the CF gRNA sequence to be transcribed to produce transformed mammalian cells;
transferring the transformed mammalian cells to the editing module; and
allowing the transformed mammalian cells to edit, allowing the transformed mammalian cells to edit, resulting in a mammalian cell population comprising cells expressing one or more CARs.

18. The automated method of claim 17, wherein the mammalian cell population comprising cells expressing one or more CARs are T cells.

19. The automated method of claim 17, wherein the mammalian cell population comprising cells expressing one or more CARs are natural killer cells.

20. The automated method of claim 17, wherein the mammalian cell population comprising cells expressing one or more CARs express a single CAR.

21. The automated method of claim 17, wherein the mammalian cell population comprising cells expressing one or more CARs express two or more CARs.

22. The automated method of claim 17, wherein the mammalian cell population comprising cells expressing one or more CARs is a cell population with individual cells expressing one or more different CARs.

23. The automated method of claim 17, wherein the mammalian cell population comprising cells expressing one or more CARs are autologous.

24. The automated method of claim 17, wherein the mammalian cell population comprising cells expressing one or more CARs are allogeneic.

25. The automated method of claim 17, wherein the mammalian cell population comprising cells expressing one or more CARs are used to treat a human patient.
